(19) United States
(12) Patent Application Publication
(10) Pub. No.: US 2024/0360273 A1
(43) Pub. Date: Oct. 31, 2024

(54) VALORISATION OF LACTIC ACID STREAM IN THE PRODUCTION PROCESS OF POLYLACTIC ACID

(71) Applicant: FUTERRO S.A., Escanaffles (BE)

(72) Inventors: Philippe COSZACH, Escanaffles (BE); Julien MORLOT, Escanaffles (BE)

(21) Appl. No.: 18/683,148

(22) PCT Filed: Aug. 23, 2022
(86) PCT No.: PCT/EP2022/073508

§ 371 Date: Feb. 12, 2024

(30) Foreign Application Priority Data
Aug. 26, 2021 (EP) 21193241.3

(51) Int. Cl. C08G 63/08; C07C 51/43; C08J 11/02

(52) U.S. Cl. CPC C08G 63/08; C07C 51/43; C08J 11/02; C07B 2200/13; C08J 2367/04

(57) ABSTRACT

The present invention discloses a valorisation method of a flux containing undesired lactic acid (ester(s)) in the production process of polylactic acid. the lactic acid and/or the lactic acid ester(s) obtained by recycling during the various stage of the production of poly lactic acid are forming a recycle stream, 0 to 100% by weight of which are subjected to a treatment in order to selectively separate a fraction containing L-lactic acid and/or L-lactic acid ester(s) from a fraction containing D-lactic acid and/or D-lactic acid ester(s); and 100 to 0% by weight of said recycle stream are used as a base for the synthesis of molecules insensitive to the optical isometry D or L of lactic acid and/or of lactic acid ester(s).

### TECHNICAL FIELD

The present invention relates to a method of valorisation of a flux containing undesired D- or L-lactic acid and/or undesired D- or L-lactic acid ester(s) in the production process of L- or D-polylactic acid. In particular, the present invention relates to a method wherein the lactic acid and/or the lactic acid ester(s) obtained by recycling during the various stage of the production of polylactic acid are forming a recycle stream containing undesired D- or L-lactic acid and/or undesired D- or L-lactic acid ester(s), 0 to 100% by weight of which are subjected to a treatment in order to selectively separate a fraction containing L-lactic acid and/or L-lactic acid ester(s) from a fraction containing D-lactic acid and/or D-lactic acid ester(s); and 100 to 0% by weight of said recycle stream are used as a base for the synthesis of molecules insensitive to the optical isometry D or L of lactic acid and/or of lactic acid ester(s).

### BACKGROUND ART

The last decades, biodegradable polymers have gained worldwide interest because of their capability to decompose in a natural environment. Examples of biodegradable polymers of interest are, without being limited thereto, aliphatic polyesters such as polylactic acid, polyhydroxybutyrate and polycaprolactone. In particular polylactic acid (PLA) has been studied because, next to being biodegradable, it is produced from lactic acid, which can be obtained from a raw material derived from a living body or a derivative thereof, such as fermented plant starch such as from corn, cassava, sugarcane or sugar beet pulp. Hence, PLA is considered as a high-safety and environmentally friendly polymer material. Nowadays, PLA can be found in various applications and forms, such as (stretched) films, fibres, injection molded products and filaments for 3D-printing.

Two main methods for the production of PLA from lactic acid are known. The first method is a direct polymerisation by polycondensation of lactic acid. Disadvantages of this method are the need for a solvent and the difficulties encountered in removing the water produced upon polycondensation out of the reaction medium.

The second method is a multi-step process, comprising oligomerisation of lactic acid, cyclization of the oligomers into lactide, and polymerisation by ring opening of the lactide, thereby obtaining polylactic acid. One of the disadvantages of this method is that in each step by-products are obtained. Consequently, the efficiency of the polylactic acid production process is estimated to be around 50 mol %.

However, several of these by-products can be recycled, in particular by reuse in the PLA production process.

WO95/09879 discloses a process for the continuous production of substantially purified lactide and lactide polymers from lactic acid or an ester of lactic acid, including the steps of forming crude polylactic acid, preferably in the presence of a catalyst means in the case of the ester of lactic acid, to form a condensation reaction by-product and polylactic acid, and depolymerizing the polylactic acid in a lactide reactor to form crude lactide, followed by subsequent purification of the crude lactide in a distillation system, wherein the purified lactide can be a meso-lactide depleted flow of L-lactide and D-lactide. A purified lactide is then polymerized to form lactide polymers. By-products of the various reaction steps can be recycled.

With processes such as the one disclosed in WO95/09879, a total efficiency of about 75 mol % can be obtained. However, an efficiency of 75 mol % is considered insufficient for rendering the process applicable on industrial scale.

WO2015/086613 discloses a process wherein several by-products of the PLA production via the method comprising oligomerisation, cyclisation and polymerisation by ring opening, are reused in the PLA synthesis. Recycled by-products are in particular unreacted lactic acid, low weight and/or unreacted oligomers and lactide are reused. Part of the by-products is converted into lactic acid prior to reuse, by means of steps of transesterification, distillation and hydrolysis.

WO2014/180836 discloses a lactide purification process for use in for example the synthesis of L-polylactic acid. A purified lactide stream comprising L-lactide and meso-lactide is obtained from a crude lactide comprising L-lactide, D-lactide and meso-lactide. The lactide purification process comprises a first distillation step, a melt crystallization step and a second distillation step. The lactide purification process allows to reduce the amount of unwanted by-products due to the recovery of a large portion of the L-lactide and at least part of the meso-lactide in the crude lactide.

Lactic acid has two optically active enantiomers, D-lactic acid (or R-lactic acid) and L-lactic acid (or S-lactic acid). Consequently, three stereoisomers of lactide can be produced, namely L-lactide, D-lactide, and meso-lactide. The three stereoisomers of lactide can result in three stereochemical forms of polylactic acid (PLA), namely: D-PLA, L-PLA and a racemic mixture called D,L-PLA. The three stereochemical forms of polylactic acid comprise different properties. It is for example known that the presence of D enantiomers in L-PLA results in a decrease of the glass transition temperature.

It is known that all of production processes of PLA generate by-products and induce racemization reactions. In a process for the production of L-PLA, this results in increasing the-D-enantiomer content in the system and vice-versa, in a process for the production of D-PLA, it is the -L- enantiomer content which is increased.

The accumulation of -D- or -L- enantiomer (depending on whether it is the production of L-PLA or D-PLA) in the system has an impact, not on the chemical purity of the PLA, but on its optical purity.

Depending on the application of the polylactic acid, PLA comprising a high amount of one of the stereochemical forms is preferably used, or rather a mixture having specific amounts of the enantiomers. State of the art methods allow to obtain rather “pure” polylactic acid, i.e. polylactic acid substantially comprising L-polylactic acid and small amounts (e.g. 4% by weight or less, based on the total weight of PLA) of D,L-PLA. However, these methods generate several residual streams comprising compounds that are considered waste, thereby limiting the overall efficiency or the overall yield of the process. It is well understood that in the production of L-polylactic acid, the residual streams comprise substantial amounts of compounds having a D-enantiometry (optical isometry), such as D-lactic acid, D-lactic acid oligomers, D-lactides, D,L-lactic acid oligomers, meso-lactides and D,L-PLA. Therefore, recycling these streams into lactic acid will contribute to increasing the content of the D-enantiomer, consequently leading to a loss of yield; moreover, if we allow to accumulate, it will lead to a drastic decrease in the optical purity of the resulting L-PLA.

By reciprocity, it is evident that the residual streams from the production of D-PLA include the same compounds but having the optical isometry -L-.

Hence, there is a need for the valorisation of these unwanted by-products. By valorising at least part of the unwanted residual products, less residual products are considered waste, hence, the efficiency of the production of PLA is increased.

### SUMMARY OF THE INVENTION

It is an object of the present invention to overcome one or more of the above drawbacks. It is an aim of the invention to provide a valorisation of undesired by-products containing undesired lactic acid and/or undesired lactic acid ester(s) in the production process of polylactic acid.

According to an aspect of the invention, there is provided a method of valorisation of a flux containing undesired D- or L-lactic acid and/or one or more undesired D- or L-lactic acid esters in the production process of L- or D-polylactic acid.

According to the process of the present invention, lactic acid and/or lactic acid ester(s) obtained by recycling during the various stages of the production of PLA are forming a recycle stream, 0 to 100% of which are subjected to a treatment in order to selectively separate a fraction containing L-lactic acid and/or L-lactic acid ester(s) from a fraction containing D-lactic acid and/or D-lactic acid ester(s); and 100 to 0% are used as a base for the synthesis of molecules insensitive to the optical isometry D or L of lactic acid and/or of lactic acid ester(s).

Advantageously, the treatment in order to selectively separate a fraction containing L-lactic acid and/or L-lactic acid ester(s) from a fraction containing D-lactic acid and/or D-lactic acid ester(s) comprises the following steps:
i. Concentration in an evaporator under operating condition enabling to obtain a concentrated lactic acid solution having a total acid content of at least 90% by weight and a content of monomeric lactic acid of at least 80% by weight based on the lactic acid concentrate,
ii. Crystallization of the concentrated lactic acid in one or more stages thereby obtaining a lactic acid with a higher chiral purity

More advantageously, the treatment in order to selectively separate a fraction containing L-lactic acid and/or L-lactic acid ester(s) from a fraction containing D-lactic acid and/or D-lactic acid ester(s) comprises the steps of:
i. Concentration and distillation in evaporators under operating condition enabling to obtain a concentrated lactic acid solution having a total acid content of at least 90% by weight and a content of monomeric lactic acid of at least 80% by weight based on the lactic acid concentrate,
ii. Crystallization of the concentrated lactic acid in one or more stages thereby obtaining a lactic acid with a higher chiral purity

Advantageously, the concentration step takes place at reduced pressure and in any reactor/evaporator able to concentrate the lactic acid solution by keeping a high content of monomeric lactic acid in the concentrated solution as for example a falling film evaporator, a thin film evaporator or a short path evaporator.

Advantageously, the distillation step when applied takes place in any equipment able to distillate, under reduced pressure, the lactic acid solution by keeping a high content of monomeric lactic acid in the distilled solution as for example a falling film evaporator, a thin film evaporator or a short path evaporator.

Advantageously, the crystallization step takes place in one or more cooling crystallizers, one or more evaporation crystallizers and/or one or more adiabatic crystallizers.

Advantageously, the molecules insensitive to the optical isometry D or L are selected from the group consisting of acrylic acid, acrylic ester, acetaldehyde, 2,3-pentanedione, pyruvic acid, and 1,2-propanediol.

The base for the synthesis of such molecules can comprise up to 100% of lactic acid and/or lactic acid esters obtained as residual product or by-product in the production of polylactic acid.

In other terms the process of the invention enables to increase the content of the wanted enantiomer L- or D- together with using the remaining unwanted enantiomer D- or L- to produce products insensitive to the L- or D- content of the lactic acid.

The inventors have noticed that by means of the methods of the invention, the efficiency and the flexibility of the production of PLA can be increased significantly compared to known processes comprising recycling of (part of) the by-products. With “production flexibility of the PLA” it is meant in the present invention the capability to obtain PLA having the requested or required properties, such as Tg and Tm.

Further, methods of the invention provide for recycling or reuse of the remaining portion of the residual streams, so that the overall efficiency is increased and thus the amount of waste products is reduced when compared to prior art methods. Consequently, the invention provides methods that can be used and implemented on an industrial scale.

### DETAILED DESCRIPTION

The applicant has now found that lactic acid and/or lactic acid ester(s) obtained by recycling during the various stages of the production of PLA and forming a recycle stream could be subjected, to a treatment in order to selectively separate a fraction containing L-lactic acid and/or L-lactic acid ester(s) from a fraction containing D-lactic acid and/or D-lactic acid ester(s); and be used as a base for the synthesis of molecules insensitive to the optical isometry D or L of lactic acid and/or of lactic acid ester(s).

More precisely, the present invention relates to a method of valorisation of either a flux containing undesired D-lactic acid and/or undesired D-lactic acid ester(s) in the production process of L-polylactic acid; or a flux containing undesired L-lactic acid and/or undesired L-lactic acid ester(s) in the production process of D-polylactic acid.

In particular, the present invention relates to a method wherein 0 to 100% by weight of a recycle stream containing undesired D-lactic acid and/or undesired D-lactic acid ester(s) are subjected to a treatment in order to selectively separate a fraction rich in L-lactic acid and/or L-lactic acid ester(s) from a fraction containing most of the D-lactic acid and/or D-lactic acid ester(s) thereby improving the efficiency of the production of L-PLA; and 100 to 0% by weight of said recycle stream are used as a base for the synthesis of molecules insensitive to the optical isometry D or L.

By reciprocity, the present invention relates to a method wherein 0 to 100% by weight of a recycle stream containing undesired L-lactic acid and/or undesired L-lactic acid ester(s) are subjected to a treatment in order to selectively separate a fraction rich in D-lactic acid and/or D-lactic acid ester(s) from a fraction containing most of the L-lactic acid and/or L-lactic acid ester(s) thereby improving the efficiency of the production of D-PLA; and 100 to 0% by weight of said recycle stream are used as a base for the synthesis of molecules insensitive to the optical isometry D or L.

According to the invention, 0 to 100% by weight of the lactic acid and/or lactic acid ester(s) obtained by recycling during the production of PLA are subjecting, to a concentration step, under operating condition enabling to obtain a concentrated lactic acid solution having a total acid content of at least 90% by weight and a content of monomeric lactic acid of at least 80% by weight based on the lactic acid concentrate.

The concentrated lactic acid obtained at the concentration step is subjecting to a crystallization step in one or more stages, thereby obtaining a lactic acid with a higher chiral purity.

The concentration step consists in concentrating, rapidly and at low temperature, the lactic acid solution. A preferred approach envisages conducting this concentration under reduced pressure, which is maintained between 50 and 500 mbar absolute, preferably between 50 and 250 mbar, in order to ensure that the water is extracted at a temperature which is as low as possible.

Preferably, the concentration step takes place in any reactor/evaporator able to concentrate the lactic acid solution by keeping a high content of monomeric lactic acid in the concentrated solution as for example a falling film evaporator, a thin film evaporator or a short path evaporator.

In a preferred embodiment of the present invention, the concentration step could be followed by a distillation step of the lactic acid in order to ensure a high content of monomeric lactic acid in the distilled solution. This operation is also performed rapidly and at low temperature and pressure. The distillation is preferably carried out at a pressure of from 0.1 to 20 mbar and a temperature of from 100 to 200° C. and preferably at a pressure of from 0.2 to 10 mbar and a temperature of from 110 to 150° C.

Preferably, the distillation step takes place in any equipment able to distillate, under reduced pressure, the lactic acid solution by keeping a high content of monomeric lactic acid in the distilled solution as for example a falling film evaporator, a thin film evaporator or a short path evaporator.

Starting from the concentrated, optionally distilled, lactic acid solution, the one or more crystallization steps can be carried out using any known crystallization techniques such as melt crystallization (cooling crystallization), evaporation crystallization or adiabatic crystallization.

Preferably, the crystallization step takes place in a system selected from one or more cooling crystallizers, one or more evaporation crystallizers, one or more adiabatic crystallizers.

Then the crystallized lactic acid is separate from the mother liquor by any known liquid-solid separation techniques.

Preferably, the crystallized lactic acid is separated from the mother liquor by centrifugation, decantation or filtration.

The mother liquor from the one or more crystallizations can optionally be recycled either at the level of the concentration stage, or at the crystallization stage.

Advantageously, the obtained lactic acid crystals are diluted and dissolved with water and the obtained solution has a higher chiral purity than before the treatment.

Advantageously, the obtained lactic acid has after at least one crystallization a chiral purity higher than 90% by weight, preferentially higher than 95% by weight, preferentially higher than 97% by weight, preferentially higher tant 98% by weight, or preferentially higher than 99% by weight. Although the above-mentioned ranges are preferential, it is understood when we are mainly looking to reduce the fraction of D(−) which disturbs the recycling of the different streams in the production of L-polylactic acid (or to reduce the fraction of L(+) in the production of D-polylactic acid), that lower values also make it possible to achieve the object of the present invention, because this stream will then be diluted with a large proportion of a stream rich in L(+) (or a stream rich in D(−)) during recycling.

Advantageously, the obtained chiral purified lactic acid or lactic acid solution can be recycled in the production of polylactic acid and/or in the production of copolymers comprising polylactic acid.

Advantageously, the mother liquor, purified or not, can be used, at least partially, as a base for the synthesis of molecules insensitive to the optical isometry D or L of lactic acid and/or lactic acid ester(s).

The mother liquor can be purified, if necessary, by any known purification techniques such as nanofiltration, microfiltration, activated carbon, resins, . . .

Advantageously, 100 to 0% by weight of the lactic acid and/or lactic acid ester(s) obtained by recycling during the production of PLA are used as a base for the synthesis of molecules insensitive to the optical isometry D or L of lactic acid and/or lactic acid ester(s).

Preferably, such molecules insensitive to the optical isometry D or L are selected from the group consisting of acrylic acid, acrylic ester, acetaldehyde, 2,3-pentanedione, pyruvic acid, and 1,2-propanediol.

The base for the synthesis of such molecules can comprise up to 100% of lactic acid and/or lactic acid esters obtained as residual product or by-product in the production of polylactic acid. For example, said molecules insensitive to the optical isometry D or L can be synthesised from a mixture of such residual lactic acid (esters) and lactic acid (esters) obtained from another source, such as “virgin” lactic acid obtained by fermentation of (plant) starch. Preferably, the lactic acid used for the synthesis of such molecules comprises between 10% and 100% by weight of residual lactic acid (esters) based on the total weight of lactic acid (esters) used in the synthesis, such as at least 10% by weight, at least 20% by weight, preferably at least 25% by weight, more preferably at least 50% by weight, for example between 50% and 100% by weight.

The present invention is further illustrated by means of the following non-limiting examples.

### EXAMPLES

### Example 1

A lactic acid obtained by recycling during the various stages of the production of L-PLA and comprising 9.1% by weight of D lactic acid based on the total weight of the lactic acid, was concentrated in an evaporator at 250 mbar. The obtained lactic acid had a concentration of 91.9% by weight, a content of monomeric lactic acid of 87.4% by weight based on the concentrated lactic acid and a L(+) content of 90.9% by weight.

The obtained lactic acid was then subjected to two successive stages of cooling crystallisation at a temperature of respectively 9° C. and 16° C.

The crystal slurry obtained was separated by centrifugation. The obtained lactic acid had a L(+) content of 97.6% by weight and the mother liquor had a total acid content of 89.5% by weight and a L(+) content of 87.9% by weight.

This chiral purified L-lactic acid was recycled in the process for the production of L-polylactic acid and the mother liquor was recycled at the concentration step.

### Example 2

A lactic acid obtained by recycling during the production of D-PLA and comprising 30% of L-lactic acid based on the total weight of the lactic acid, was used for the synthesis of pyruvic acid. First, the lactic acid was transformed into ethyl lactate by a known process, then the feed comprising ethyl lactate was introduced at the top of a tubular reactor in order to be vaporized. The vapour was then passed through three layers of catalyst made of silver crystal beads. The reaction was carried out at a temperature of 550° C. and at a pressure of 1.2 bar. The gaseous reaction mixture was then cooled down to 20° C. and washed with water, and the ethyl pyruvate was recovered in good yield (72%). The ethyl pyruvate was of equivalent quality than an ethyl pyruvate produced from non-recycled ethyl lactate with a similar conversion and selectivity.

### Example 3

A lactic acid obtained by recycling during the production of L-PLA comprising undesired D-lactic acid was concentrated in an evaporator at 250 mbar and then distilled in a thin film evaporator at 10 mbar. The obtained lactic acid had a concentration of 96.1% by weight, a content of monomeric lactic acid of 94.1% by weight based on the concentrated lactic acid and a L(+) content of 85.6% by weight.

The obtained lactic acid was then subjected to two successive stages of cooling crystallisation at a temperature of respectively 5° C. and 14° C.

The crystal slurry obtained was separated by centrifugation. The obtained lactic acid had an L(+) content of 95.3% by weight.

This chiral purified L-lactic acid was recycled partially in the process for the production of L-polylactic acid and the mother liquor, containing D-lactic acid, was used to prepare 2,3-pentanedione by condensation of this recycled D-lactic acid, in the presence of a sodium phosphate catalyst deposited on a silica/alumina support.

A 50% aqueous solution of lactic acid was prepared with the mother liquor which was brought into contact with the catalyst described above at a temperature of 300° C. and under a pressure of 5 MPa.

2,3-pentanedione was obtained in good yield (76%), it was of equivalent quality than a 2,3-pentanedione produced from non-recycled lactic acid with similar conversion and selectivity.

### Example 4

A lactic acid obtained by recycling during the various stages of the production of L-PLA is forming a recycle stream comprising 28.8% of D-lactic acid based on the total weight of the lactic acid.

70% by weight of said recycle stream comprising undesired D-lactic acid was concentrated in an evaporator at 250 mbar. The concentrated lactic acid solution had a concentration of 96.2% by weight and a L(+) content of 71.2% by weight based on the total weight of the concentrated lactic acid solution.

The concentrated lactic acid solution was then subjected to a cooling crystallisation at a temperature of 5° C.

The crystal slurry obtained was separated by centrifugation. The obtained crystallized lactic acid had a total acid content of 97.8% by weight and a L(+) content of 84.2% by weight and the mother liquor had a total acid content of 92.7% by weight and a L(+) content of 52.3% by weight.

The mother liquor and the remaining 30% by weight of said recycle stream comprising undesired D-lactic acid was used for the synthesis of pyruvic acid. First, the lactic acid was transformed into ethyl lactate by a known process, then the feed comprising ethyl lactate was introduced at the top of a tubular reactor in order to be vaporized. The vapour was then passed through three layers of catalyst made of silver crystal beads. The reaction was carried out at a temperature of 550° C. and at a pressure of 1.2 bar. The gaseous reaction mixture was then cooled down to 20° C. and washed with water, and the ethyl pyruvate was recovered in good yield (73%). The ethyl pyruvate was of equivalent quality than an ethyl pyruvate produced from non-recycled ethyl lactate with a similar conversion and selectivity.

1. A method of valorization of a flux containing undesired lactic acid and/or one or more undesired lactic acid esters in the production process of polylactic acid, wherein the lactic acid and the one or more lactic acid esters obtained by recycling during the various stage of the production of PLA are forming a recycle stream, wherein some or all of the recycle stream is subjected to a treatment in order to selectively separate a fraction containing L-lactic acid and/or L-lactic acid ester(s) from a fraction containing D-lactic acid and/or D-lactic acid ester(s), said treatment comprising the following steps:
(i) concentrating the recycle stream in one or more evaporator(s) under operating condition to obtain a concentrated lactic acid solution having a total acid content of at least 90% by weight and a content of monomeric lactic acid of at least 80% by weight based on the lactic acid concentrate;
(ii) crystallization of the concentrated lactic acid in one or more stage(s) to obtain a lactic acid with a higher chiral purity;
using some or all of said recycle stream a base for the synthesis of molecules insensitive to the optical isometry D or L of lactic acid and/or of lactic acid ester(s).

2. The method according to claim 1, wherein the one or more evaporator(s) comprise a falling film evaporator, a thin film evaporator, or a short path evaporator.

3. The method according to claim 1, wherein the concentration is conducted under reduced pressure, which is maintained between 50 and 500 mbar absolute, and at low temperature.

4. The method according to claim 11, wherein the distillation is carried out at a pressure of from 0.1 to 20 mbar and a temperature of from 100 to 200° C.

5. The method according to claim 1, wherein the crystallization is carried out in one or more cooling crystallizers, one or more evaporation crystallizers and/or one or more adiabatic crystallizers.

6. The method according to claim 1, wherein the crystallized lactic acid is separate from the mother liquor using a solid-liquid separation.

7. The method according to claim 1, wherein the obtained chiral purified lactic acid or lactic acid solution is recycled in the production of polylactic acid and/or in the production of copolymers comprising polylactic acid.

8. The method according to claim 1, wherein a mother liquor from the one or more crystallization stage(s) is recycled at the concentration stage.

9. The method according to claim 8, wherein the mother liquor from the one or more crystallization stage(s) is purified before recycling.

10. A method of valorization of a flux containing undesired D-lactic acid and/or one or more undesired D-lactic acid esters in the production process of L-polylactic acid, wherein the D-lactic acid and the one or more D-lactic acid ester(s) obtained by recycling during the various stage of the production of L-PLA are forming a recycle stream subjected to a treatment in order to selectively separate a fraction rich in L-lactic acid and/or L-lactic acid ester(s) from a fraction containing most of the D-lactic acid and/or D-lactic acid ester(s), said treatment comprising the following steps:
(i) concentration in one or more evaporator(s) to obtain a concentrated lactic acid solution having a total acid content of at least 90% by weight and a content of monomeric lactic acid of at least 80% by weight based on the lactic acid concentrate;
(ii) crystallization of the concentrated lactic acid in one or more stage(s) to obtain a lactic acid with a higher chiral purity;
wherein some or all of said recycle stream is used as a base for the synthesis of molecules selected from the group consisting of acrylic acid, acrylic ester, acetaldehyde, 2,3-pentanedione, pyruvic acid, and 1,2 propanediol.

11. The method of claim 1, wherein concentrating the recycle stream in one or more evaporator(s) further comprises distillation.

12. The method of claim 1, wherein the molecules are selected from the group consisting of acrylic acid, acrylic ester, acetaldehyde, 2,3-pentanedione, pyruvic acid, and 1,2 propanediol.
